(19) United States
(12) Patent Application Publication
(10) Pub. No.: US 2010/0196282 A1
(43) Pub. Date: Aug. 5, 2010

(54) OPTICAL IMAGING AGENTS

(76) Inventors: Robert James Domett Nairne, Amersham (GB); Andrew John Healey, Oslo (NO)

Correspondence Address: GE HEALTHCARE BIO-SCIENCES CORP.;PATENT DEPARTMENT, 101 CARNEGIE CENTER, PRINCETON, NJ, 08540, US

(21) Appl. No.: 12/671,036

(22) PCT Filed: Jul. 29, 2008
(86) PCT No.: PCT/EP2008/059942

§ 371 Date: Jan. 28, 2010

Related U.S. Application Data
(60) Provisional application No. 60/976,817, filed on Oct. 2, 2007.

(30) Foreign Application Priority Data
Sep. 28, 2007 (GB) 0718957.4

(51) Int. Cl. A61K 49/00; C07K 2/00; C07H 21/04; C07H 21/02

(52) U.S. Cl. 424/9.6; 424/9.1; 530/300; 435/177; 536/23.1

(57) ABSTRACT

The present invention relates to imaging agents suitable for in vivo optical imaging, which comprise conjugates of benzopyrylium dyes with biological targeting moieties, such as peptides Also disclosed are pharmaceutical compositions and kits, as well as in vivo imaging methods.

### FIELD OF THE INVENTION

The present invention relates to imaging agents suitable for in vivo optical imaging, which comprise conjugates of benzopyrylium dyes with biological targeting moieties, such as peptides. Also disclosed are pharmaceutical compositions and kits, as well as in vivo imaging methods.

### BACKGROUND TO THE INVENTION

U.S. Pat. No. 6,750,346 discloses laser-compatible near-infrared (NIR) markers dyes of formulae A, B or C:

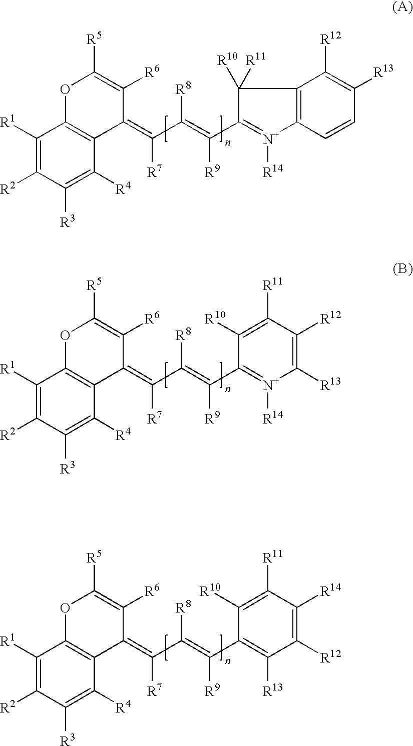

wherein:
n is 1, 2 or 3;
R¹ to R¹⁴ are the same or different and are chosen from H, Cl, Br; an aliphatic or mononuclear aromatic group, of up to 12 carbon atoms which may contain as a substituted group in addition to C and H, up to 4 oxygen atoms and 0, 1 or 2 nitrogen atoms or a sulfur atom or a sulfur and a nitrogen atom or represent an amino function, having a nitrogen atom to which there is bound, H or at least one substituent having up to 8 carbon atoms, said substituent selected from the group consisting of C, H and up to two sulfonic acid groups.

The dyes of U.S. Pat. No. 6,750,346 are chosen such that preferably at least one of R¹ to R¹⁴ contains a solubilising or ionisable group. Such groups are said to include: cyclodextrin, sugar, SO₃⁻, PO₃²⁻, CO₂⁻ or NR₃⁺. U.S. Pat. No. 6,750,346 teaches that the dyes, as well as systems derived from them (conjugates) can be used in optical, especially in fluorescence optical qualitative and quantitative determination methods for the diagnosis of cell properties, in biosensors (point-of-care measurements), exploration of the genome and in miniaturisation technology. Typical such applications being in the fields of: cytometry, cell sorting, fluorescence correlation spectroscopy (FCS), ultra-high throughput screening (UHTS), multicolour fluorescence in situ hybridization (FISH) and in microarrays (gene and protein chips).

U.S. Pat. No. 6,924,372 discloses asymmetrical polymethine dyes of formula D or E:

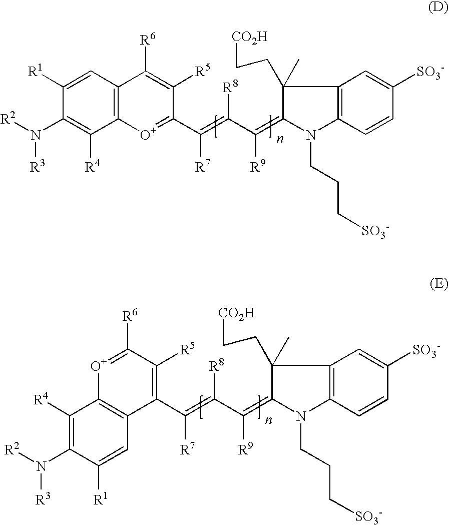

where:
n is 0, 1, 2 or 3;
R¹-R⁹ are the same or different and may be H, alkyl-, tert-alkyl, aryl-, carboxyaryl-, dicarboxyaryl, heteroaryl-, cycloalkyl-, heterocycloalkyl-, alkyloxy-, alkylmercapto- (with “alkyl” and “cycloalkyl” also including olefin linkage residues), aryloxy-, arylmercapto-, heteroaryloxy-, heteroarylmercapto-, hydroxy-, nitro- or cyano residues and R¹ and R², R² and R³, R³ and R⁴, R⁵ and R⁷ can form one or more aliphatic, heteroaliphatic or aromatic ring.

At least one of the R¹-R⁹ substituents of U.S. Pat. No. 6,924,372 may optionally be a solubilising or ionising substituent (eg. SO₃⁻, PO₃²⁻, CO₂H, OH, NR₃⁺, cyclodextrin or sugar), or may optionally be a reactive group (eg. isothiocyanate, hydrazine, active ester, maleimide or iodacetamide) permitting covalent linkage of the dye to another molecule. The dyes of formulae D and E are said to be useful in diagnosing cell characteristics or biosensors, typically cytometry and cell sorting.

Lisy et al [J. Biomed. Optics, 11(6) 064014 (2006)] disclose a method of diagnosis of peritonitis using near-infrared optical imaging and labelled monocytes or macrophages. The monocytes-macrophages could be labelled in vitro with the dye DY-676 (Dyomics GmbH). Administration of the dye DY-676 itself in an animal model of peritonitis in vivo led to increased fluorescence in the area of peritonitis. The authors concluded that monocyte-macrophage labelling had occurred in vivo.

Lisy et al [Invest. Radiol., 42(4) 235-241 (2007)] disclose bimodal (MRI and optical) contrast agents which comprise nanoparticles labelled with fluorescent magnetosomes. The fluorescent magnetosome nanoparticles were used to label macrophages by a process of phagocytosis. The dye used to label the magnetosomes was again DY-676.

The Dyomics GmbH website (www.dyomics.com) includes an image courtesy of I. Hilger (FSU Jena) entitled “Visualisation of Arthritis in a Rat by Accumulation of DY-676 in Joints”. No further details are given.

WO 2007/139815 discloses imaging and therapeutic methods involving progenitor cells. Conjugates of the formula shown are disclosed:

A_{B}-X
where:
   A_{B} comprises a vitamin or analog that binds to CD133⁺ Flk1⁺ endothelial progenitor cells;
   X is a quantifiable marker.

The quantifiable marker can be eg. a radioactive probe or a fluorescent probe. Suitable fluorescent probes are stated to be: fluorescein, rhodamine, Texas Red, phycoerythrin, Oregon Green, Alexa Fluor 488 . . . , Cy3, Cy5, Cy7, and the like. Example 30 of WO 2007/139815 discloses a single benzopyrylium dye (DyLight™ 680) conjugated to folate via a 5-mer peptide linker (Asp-Arg-Asp-Asp-Cys).

### THE PRESENT INVENTION

The present invention provides imaging agents suitable for in vivo optical imaging, which comprise a specific class of benzopyrylium dye conjugated to a biological targeting moiety (BTM). The present inventors have identified sulfonated benzopyrylium dyes which are suitable for in vivo optical imaging applications as part of such covalently-bonded BTM conjugates.

The benzopyrylium dyes (Bzp^{M}) of the present invention possess a combination of properties which make them useful for in vivo optical imaging applications:
(i) capability of conjugation to biological targeting molecules (BTM);
(ii) water solubility;
(iii) absorption and emission in the red, far red or near infra-red portion of the electromagnetic spectrum;
(iv) high extinction coefficients;
(v) low blood plasma protein binding;
(vi) high photostability and brightness;
(vii) high stability of dye and dye-BTM conjugate in blood;
(viii) rapid clearance from the blood in vivo;
(ix) lack of potentially dangerous metabolites (by Meteor/Derek analyses).

### DETAILED DESCRIPTION OF THE INVENTION

In a first aspect, the present invention provides a pharmaceutical composition which comprises an imaging agent suitable for in vivo optical imaging of the mammalian body, together with a biocompatible carrier, said composition being in a form suitable for mammalian administration, wherein said imaging agent comprises a conjugate of Formula I:

[BTM]-(L)ₙ-Bzp^{M} (I)
where:
BTM is a biological targeting moiety;
n is an integer of value 0 or 1;
L is a synthetic linker group of formula -(A)ₘ- wherein m is an integer of value 1 to 20, and each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block; wherein each R is independently chosen from H, C₁₋₄ alkyl, C₂₋₄ alkenyl, C₂₋₄ alkynyl, C₁₋₄ alkoxyalkyl or C₁₋₄ hydroxyalkyl;
Bzp^{M} is a benzopyrylium dye of Formula II:

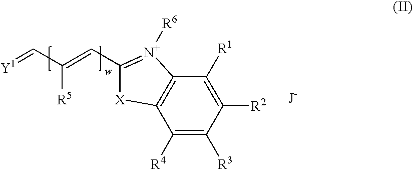

where:
   Y¹ is a group of Formula Y^{a} or Y^{b}

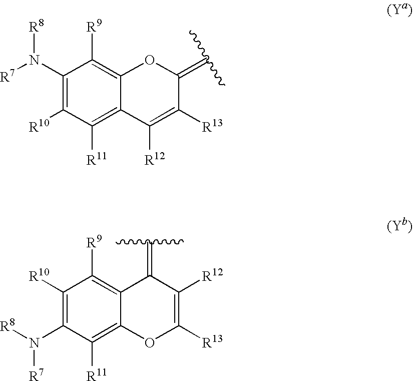

R¹-R⁴ and R⁹-R¹³ are independently selected from H, —SO₃M¹, Hal, R^{a} or C₃₋₁₂ aryl, where each M¹ is independently H or B^{c}, and B^{c} is a biocompatible cation;
R⁵ is H, C₁₋₄ alkyl, C₁₋₆ carboxyalkyl, C₃₋₁₂ arylsulfonyl, Cl, or R⁵ together with one of R⁶, R¹⁴, R¹⁵ or R¹⁶ may optionally form a 5- or 6-membered unsaturated aliphatic, unsaturated heteroaliphatic or aromatic ring;
R⁶ and R¹⁶ are independently R^{a} groups;
R⁷ and R⁸ are independently C₁₋₄ alkyl, C₁₋₄ sulfoalkyl or C₁₋₆ hydroxyalkyl or optionally together with one or both of R⁹ and/or R¹⁰ may form a 5- or 6-membered N-containing heterocyclic or heteroaryl ring;
X is —CR¹⁴R¹⁵—, —O—, —S—, —Se—, —NR¹⁶— or —CH═CH—, where R¹⁴ to R¹⁶ are independently R^{a} groups;
R^{a} is C₁₋₄ alkyl, C₁₋₄ sulfoalkyl, carboxyalkyl or C₁₋₆ hydroxyalkyl;
w is 1 or 2;
J is a biocompatible anion;
with the proviso that Bzp^{M} comprises at least one sulfonic acid substituent chosen from the R¹ to R¹⁶ groups.

By the term “imaging agent” is meant a compound suitable for optical imaging of a region of interest of the whole (ie. intact) mammalian body in vivo. Preferably, the mammal is a human subject. The imaging may be invasive (eg. intra-operative or endoscopic) or non-invasive. The imaging may optionally be used to facilitate biopsy (eg. via a biopsy channel in an endoscope instrument), or tumour resection (eg. during intra-operative procedures via tumour margin identification).

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

The green to near-infrared region light is suitably of wavelength 500-1200 nm, preferably of wavelength 550-1000 nm, most preferably 600-800 nm. The optical imaging method is preferably fluorescence endoscopy. The mammalian body of the sixth aspect is preferably the human body. Preferred embodiments of the imaging agent are as described for the first aspect (above). In particular, it is preferred that the Bzp^{M} dye employed is fluorescent.

By the term “biocompatible carrier” is meant a fluid, especially a liquid, in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection or isotonic saline.

By the term “conjugate” is meant that the BTM, (L)ₙ group and Bzp^{M} dye are linked by covalent bonds.

Whilst the conjugate of Formula I is suitable for in vivo imaging, it may also have in vitro applications (eg. assays quantifying the BTM in biological samples or visualisation of BTM in tissue samples). Preferably, the imaging agent is used for in vivo imaging.

By the term “sulfonic acid substituent” is meant a substituent of formula —SO₃M¹, where M¹ is H or B^{c}, and B^{c} is a biocompatible cation. The —SO₃M¹, substituent is covalently bonded to a carbon atom, and the carbon atom may be aryl (ie. sulfoaryl such as when R¹ or R² is —SO₃M¹), or alkyl (ie. a sulfoalkyl group). By the term “biocompatible cation” (B^{c}) is meant a positively charged counterion which forms a salt with an ionised, negatively charged group (in this case a sulfonate group), where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “biocompatible anion” (J) is meant a negatively charged counterion which forms a salt with an ionised, positively charged group (in this case an indolinium group), where said negatively charged counterion is also non-toxic and hence suitable for administration to the mammalian body, especially the human body. The counterion (J⁻) represents an anion which is present in a molar equivalent amount, thus balancing the positive charge on the Bzp^{M} dye. The anion (J) is suitably singly- or multiply-charged, as long as a charge-balancing amount is present. The anion is suitably derived from an inorganic or organic acid. Examples of suitable anions include: halide ions such as chloride or bromide; sulfate; nitrate; citrate; acetate; phosphate and borate. A preferred anion is chloride.

By the term “biological targeting moiety” (BTM) is meant a compound which, after administration to the mammalian body in vivo, is taken up selectively or localises at a particular site of said mammalian body. Such sites may for example be implicated in a particular disease state be indicative of how an organ or metabolic process is functioning. The biological targeting moiety preferably comprises: 3-100 mer peptides, peptide analogues, peptoids or peptide mimetics which may be linear peptides or cyclic peptides or combinations thereof; or enzyme substrates, enzyme antagonists or enzyme inhibitors; synthetic receptor-binding compounds; oligonucleotides, or oligo-DNA or oligo-RNA fragments.

By the term “peptide” is meant a compound comprising two or more amino acids, as defined below, linked by a peptide bond (ie. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also “Synthesis of Peptides and Peptidomimetics”, M. Goodman et al, Houben-Weyl E22c, Thieme.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Suitable enzyme substrates, antagonists or inhibitors include glucose and glucose analogues such as fluorodeoxyglucose; fatty acids, or elastase, Angiotensin II or metalloproteinase inhibitors. A preferred non-peptide Angiotensin II antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor. When the receptor-binding compound is folate, the linker group preferably does not comprise the 5-mer peptide Asp-Arg-Asp-Asp-Cys. Most preferably, the receptor-binding compound is not folate.

The benzopyrylium dye (Bzp^{M}) of Formula II is a fluorescent dye or chromophore which is capable of detection either directly or indirectly in an optical imaging procedure using light of green to near-infrared wavelength (500-1200 nm, preferably 550-1000 nm, more preferably 600-800 nm). Preferably, the Bzp^{M} has fluorescent properties.

It is envisaged that one of the roles of the linker group -(A)ₘ- of Formula I is to distance the Bzp^{M} from the binding site of the BTM. This is particularly important because the Bzp^{M} is relatively bulky, so adverse steric interactions are possible. This can be achieved by a combination of flexibility (eg. simple alkyl chains), so that the Bzp^{M} has the freedom to position itself away from the binding site and/or rigidity such as a cycloalkyl or aryl spacer which orientate the Bzp^{M} away from the binding site. The nature of the linker group can also be used to modify the biodistribution of the imaging agent. Thus, eg. the introduction of ether groups in the linker will help to minimise plasma protein binding. When -(A)ₘ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucosamine derivative of asparagine (commercially available from NovaBiochem) is one example of this:

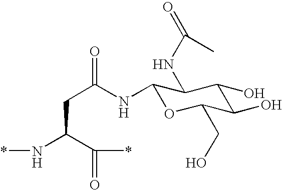

Formula I denotes that the -(L)ₙ[Bzp^{M}] moiety can be attached at any suitable position of the BTM. Suitable such positions for the -(L)ₙ[Bzp^{M}] moiety are chosen to be at positions away from that part of the BTM which is responsible for binding to the active site in vivo. The [BTM]-(L)ₙ- moiety of Formula I may be attached at any suitable position of the Bzp^{M} of Formula II. The [BTM]-(L)ₙ- moiety either takes the place of an existing substituent (eg. one of the R¹ to R¹⁶ groups), or is covalently attached to the existing substituent of the Bzp^{M}. The [BTM]-(L)ₙ- moiety is preferably attached via a carboxyalkyl substituent of the Bzp^{M}.

Suitable imaging agents of the invention are those wherein the Bzp^{M} is of Formula IIa or IIb:

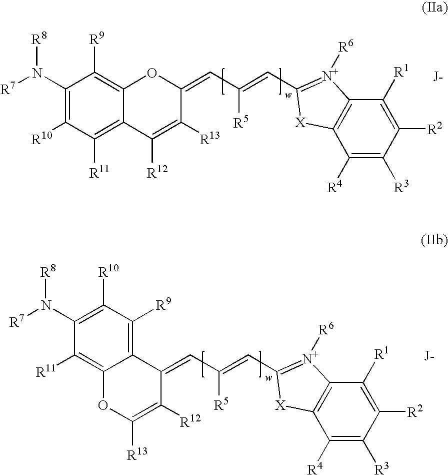

where X, w, J and R¹-R¹³ are as defined for Formula II.

When R⁵ together with one of R⁶/R¹⁴-R¹⁶ forms a 5- or 6-membered unsaturated aliphatic, unsaturated heteroaliphatic or aromatic ring, suitable such aromatic rings include: phenyl, furan, thiazole, pyridyl, pyrrole or pyrazole rings. Suitable unsaturated rings comprise at least the C═C to which R⁵ is attached.

When R⁷ and/or R⁸ together with one or both of R⁹ and/or R¹⁰ form a 5- or 6-membered N-containing heterocyclic or heteroaryl ring, suitable such rings include: thiazole, pyridyl, pyrrole or pyrazole rings or partially hydrogenated versions thereof. preferably pyridyl or dihydropyridyl.

In an alternative embodiment, the dyes of Formula IIb may optionally be chosen such that at least one of R¹ to R⁴ is F or —(CF₂)_{f}—F, where f is an integer of value 1 to 4.

The pharmaceutical composition is supplied in suitable vials or vessels which comprise a sealed container which permits maintenance of sterile integrity, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Such kits are described in the second aspect (below). Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions of the first aspect may be prepared under aseptic manufacture (ie. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical composition of the first aspect is preferably prepared from a kit, as described for the second aspect below.

### Preferred Features.

The molecular weight of the imaging agent is suitably up to 30,000 Daltons. Preferably, the molecular weight is in the range 1,000 to 20,000 Daltons, most preferably 2000 to 18,000 Daltons, with 2,500 to 16,000 Daltons being especially preferred.

The BTM may be of synthetic or natural origin, but is preferably synthetic. The term “synthetic” has its conventional meaning, ie. man-made as opposed to being isolated from natural sources eg. from the mammalian body. Such compounds have the advantage that their manufacture and impurity profile can be fully controlled. Monoclonal antibodies and fragments thereof of natural origin are therefore outside the scope of the term ‘synthetic’ as used herein.

The BTM is preferably chosen from: a 3-100 mer peptide, enzyme substrate, enzyme antagonist or enzyme inhibitor. BTM is most preferably a 3-100 mer peptide or peptide analogue. When the BTM is a peptide, it is preferably a 4-30 mer peptide, and most preferably a 5 to 28-mer peptide.

The [BTM]-(L)ₙ- moiety of Formula I is preferably attached at positions R⁵, R⁶, R¹⁴, R¹⁵ or R¹⁶ of the Bzp^{M} of Formula II, more preferably at R⁶, R¹⁴, R¹⁵ or R¹⁶ most preferably at R⁶, R¹⁴ or R¹⁵. In order to facilitate the attachment the relevant R⁵, R⁶, R¹⁴, R¹⁵ or R¹⁶ substituent is preferably C₁₋₆ carboxyalkyl, more preferably C₃₋₆ carboxyalkyl, with the carboxy group used as an active ester.

The benzopyrylium dye (Bzp^{M}) preferably has at least 2 sulfonic acid substituents, more preferably 2 to 6 sulfonic acid substituents, most preferably 2 to 4 sulfonic acid substituents. Preferably, at least one of the sulfonic acid substituents is a C₁₋₄ sulfoalkyl group. Such sulfoalkyl groups are preferably located at positions R⁶, R⁷, R⁸, R¹⁴, R¹⁵ or R¹⁶; more preferably at R⁶, R⁷, R⁸, R¹⁴ or R¹⁵; most preferably at R⁶ together with one or both of R⁷ and R⁸ of Formula II. The sulfoalkyl groups of Formula II, are preferably of formula —(CH₂)ₖSO₃M¹, where M¹ is H or B^{c}, k is an integer of value 1 to 4, and B^{c} is a biocompatible cation (as defined above). k is preferably 3 or 4.

In Formula II, w is preferably 1. R⁵ is preferably H or C₁₋₄ carboxyalkyl, and is most preferably H. X is preferably —CR¹⁴R¹⁵— or —NR¹⁶—, and is most preferably —CR¹⁴R¹⁵—.

Preferred Bzp^{M} dyes are of Formula III:

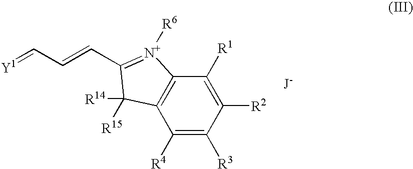

where Y¹, R¹-R⁴, R⁶, R¹⁴, R¹⁵ and J are as defined for Formula II.

Suitable dyes of Formula III are of Formula IIIc or IIIb:

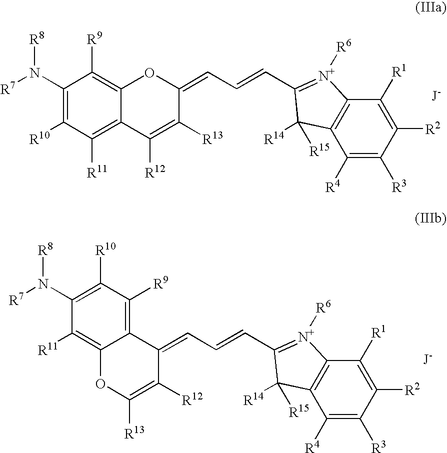

Preferred R¹-R⁴ and R⁶-R¹³ groups of Formulae III, IIIa and IIIb are as described above for formulae IIa and IIb. In Formulae III, IIIa and IIIb, R¹⁴ and R¹⁵ are preferably chosen such that one is an R^{b} group and the other is an R^{c} group. R^{b} is C₁₋₂ alkyl, most preferably methyl. R^{c} is C₁₋₄ alkyl, C₁₋₆ carboxyalkyl or C₁₋₄ sulfoalkyl, preferably C₃₋₆ carboxyalkyl or —(CH₂)ₖSO₃M¹ where k is chosen to be 3 or 4.

Preferably the dyes of Formula III have a C₁₋₆ carboxyalkyl substituent to permit facile covalent attachment to the BTM.

In Formula II or III, when R⁷ and/or R⁸ together with one or both of R⁹ and/or R¹⁰ form a 5- or 6-membered N-containing heterocyclic or heteroaryl ring, preferred such rings are pyridyl or dihydropyridyl. A preferred such Y¹ group wherein an R⁸ group has been cyclised with R¹⁰ is of Formula Y^{c}:

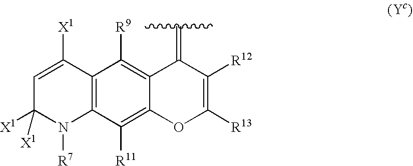

A preferred such Y¹ group wherein both R⁷ and R⁸ group have been cyclised is of Formula Y^{d}:

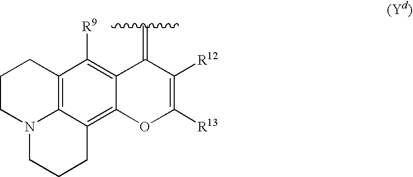

where:
R⁷, R⁹ and R¹¹-R¹³ are as defined above;
each X¹ is independently H or C₁₋₄ alkyl.

In Formula Y^{c}, it is preferred that:

each X¹ is CH₃;

### R⁹═R¹¹═H;

### R¹² is H;

R¹² is CH₃ or —C(CH₃)₃, more preferably —C(CH₃)₃.

In Formula Y^{d}, it is preferred that:

### R⁹═H;

### R¹² is H;

R¹² is preferably CH₃ or —C(CH₃)₃, more preferably —C(CH₃)₃.

It is preferred that the —NR⁷R⁸ group of Formula III is either:
(i) in open chain form, ie. the R⁷/R⁸ groups are not cyclised with one or both of R⁹/R¹⁰. Preferred such R⁷ and R⁸ groups are independently chosen from C₁₋₄ alkyl or C₁₋₄ sulfoalkyl, most preferably ethyl or C₃₋₄ sulfoalkyl;
(ii) cyclised to give a cyclic Y¹ substituent of Formula Y^{c} or Y^{d}, more preferably of Formula Y^{c}.

The open chain form (i) is most preferred.

Especially preferred dyes of Formula III are of Formula IIIc, IIId or IIIe:

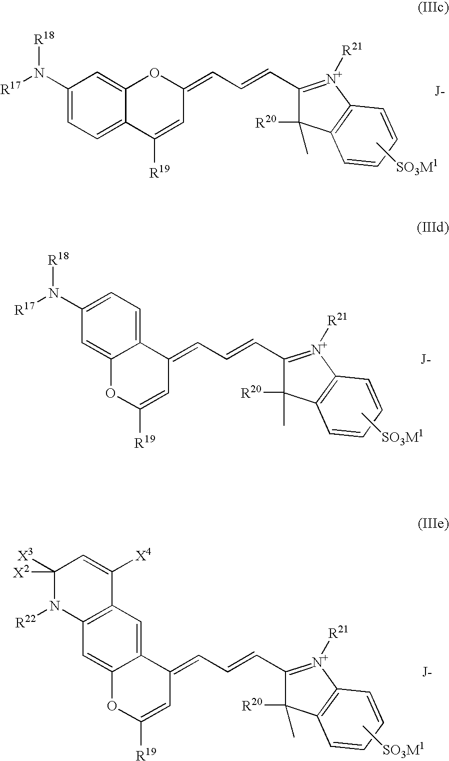

where:
M¹ and J are as defined above;
R¹⁷ and R¹⁸ are independently chosen from C₁₋₄ alkyl or C₁₋₄ sulfoalkyl;
R¹⁹ is H or C₁₋₄ alkyl;
R²⁰ is C₁₋₄ alkyl, C₁₋₄ sulfoalkyl or C₁₋₆ carboxyalkyl;
R²¹ is C₁₋₄ sulfoalkyl or C₁₋₆ carboxyalkyl;
R²² is C₁₋₄ alkyl, C₁₋₄ sulfoalkyl or C₁₋₆ carboxyalkyl;
X², X³ and X⁴ are independently H or C₁₋₄ alkyl.

The dyes of Formulae IIId, IIIe and IIIf are preferably chosen such that one or more of R²⁰-R²² is C₁₋₄ sulfoalkyl.

Preferred specific dyes of Formula IIId are DY-631 and DY-633:

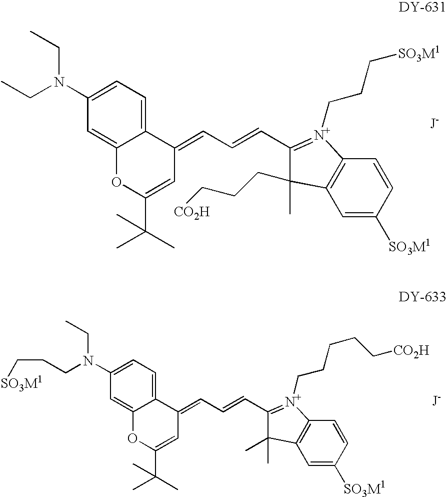

A preferred specific dye of Formula IIIe is DY-652:

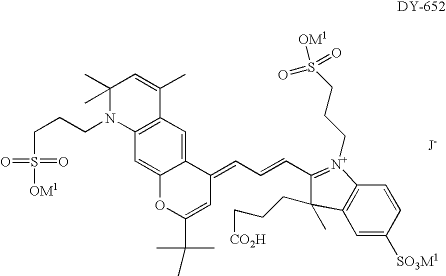

Preferred specific dyes are DY-631 and DY-652, with DY-652 being most preferred.

When the BTM is a peptide, preferred such peptides include:
somatostatin, octreotide and analogues,
peptides which bind to the ST receptor, where ST refers to the heat-stable toxin produced by E. coli and other micro-organisms;
laminin fragments eg. YIGSR, PDSGR, IKVAV, LRE and KCQAGTFALRGDPQG,
N-formyl peptides for targeting sites of leucocyte accumulation,
Platelet factor 4 (PF4) and fragments thereof,
RGD (Arg-Gly-Asp)-containing peptides, which may eg. target angiogenesis [R. Pasqualini et al., Nat. Biotechnol. 1997 June; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M. Tone et al., J. Biochem, 102, 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as:

angiotensin II
Asp-Arg-Val-Tyr-Ile-His-Pro-Phe (E. C. Jorgensen
et al, J. Med. Chem., 1979, Vol 22, 9, 1038-1044)
[Sar, Ile] Angiotensin II:
Sar-Arg-Val-Tyr-Ile-His-Pro-Ile (R. K. Turker et
al., Science, 1972, 177, 1203).
Angiolensin I:
Asp-Arg-Val-Tyr-Ile-His-Pro-Phe-His-Leu.

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase such as carboxypeptidase, metabolism of the BTM peptide at either the amino terminus or carboxy terminus. Such groups are particularly important for in vivo applications, and are well known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Suitable PEG groups are described for the linker group (L), below. Preferred such PEG groups are the biomodifiers of Formulae Bio1 or Bio2 (below). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

When either or both peptide termini are protected with an M^{IG} group, the -(L)ₙ[Bzp^{M}] moiety may optionally be attached to the M^{IG} group. Preferably, at least one peptide terminus has no M^{IG} group, so that attachment of the -(L)ₙ[Bzp^{M}] moiety at that position gives compounds of Formulae IVa or IVb respectively:

[Bzp^{M}]-(L)ₙ-[BTM]-Z² (IVa);

Z¹-[BTM]-(L)ₙ-[Bzp^{M}] (IVb);

where:
Z¹ is attached to the N-terminus of the BTM peptide, and is H or M^{IG};
Z² is attached to the C-terminus of the BTM peptide and is OH, OB^{c}, or M^{IG},
   where B^{c} is a biocompatible cation (as defined above).

In Formula IVa and IVb, Z¹ and Z² are preferably both independently M^{IG}. Preferred such M^{IG} groups for Z¹ and Z² are as described above for the peptide termini. Whilst inhibition of metabolism of the BTM peptide at either peptide terminus may also be achieved by attachment of the -(L)ₙ[Bzp^{M}] moiety in this way, -(L)ₙ[Bzp^{M}] itself is outside the definition of M^{IG} of the present invention.

The BTM peptide may optionally comprise at least one additional amino acid residue which possesses a side chain suitable for facile conjugation of the Bzp^{M}, and forms part of the A residues of the linker group (L). Suitable such amino acid residues include Asp or Glu residues for conjugation with amine-functionalised Bzp^{M} dyes, or a Lys residue for conjugation with a carboxy- or active ester-functionalised Bzp^{M} dye. The additional amino acid residue(s) for conjugation of Bzp^{M} are suitably located away from the binding region of the BTM peptide, and are preferably located at either the C- or N-terminus. Preferably, the amino acid residue for conjugation is a Lys residue.

When a synthetic linker group (L) is present, it preferably comprises terminal functional groups which facilitate conjugation to [BTM] and Bzp^{M}. Suitable such groups (Q^{a}) are described below. When L comprises a peptide chain of 1 to 10 amino acid residues, the amino acid residues are preferably chosen from glycine, lysine, arginine, aspartic acid, glutamic acid or serine. When L comprises a PEG moiety, it preferably comprises units derived from oligomerisation of the monodisperse PEG-like structures of Formulae Bio1 or Bio2:

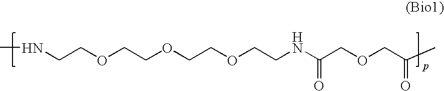

17-amino-5-oxo-6-aza-3,9,12,15-tetraoxaheptadecanoic acid of Formula Bio1
wherein p is an integer from 1 to 10. Alternatively, a PEG-like structure based on a propionic acid derivative of Formula Bio2 can be used:

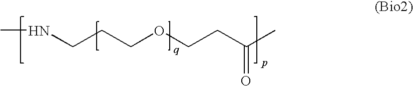

where p is as defined for Formula Bio1
and q is an integer from 3 to 15.

In Formula Bio2, p is preferably 1 or 2, and q is preferably 5 to 12.

When the linker group does not comprise PEG or a peptide chain, preferred L groups have a backbone chain of linked atoms which make up the -(A)ₘ- moiety of 2 to 10 atoms, most preferably 2 to 5 atoms, with 2 or 3 atoms being especially preferred. A minimum linker group backbone chain of 2 atoms confers the advantage that the Bzp^{M} is well-separated so that any undesirable interaction is minimised.

BTM peptides which are not commercially available can be synthesised by solid phase peptide synthesis as described in P. Lloyd-Williams, F. Albericio and E. Girald; Chemical Approaches to the Synthesis of Peptides and Proteins, CRC Press, 1997.

The imaging agents can be prepared as follows:

In order to facilitate conjugation of the Bzp^{M} to the BTM, the Bzp^{M} suitably has attached thereto a reactive functional group (Q^{a}). The Q^{a} group is designed to react with a complementary functional group of the BTM, thus forming a covalent linkage between the Bzp^{M} and the BTM. The complementary functional group of the BTM may be an intrinsic part of the BTM, or may be introduced by use of derivatisation with a bifunctional group as is known in the art. Table 1 shows examples of reactive groups and their complementary counterparts:

TABLE 1
Reactive Groups and Complementary Groups Reactive Therewith.

| Reactive Group (Q^{a}) | Complementary Groups |
|---|---|
| Activated ester | primary amino, secondary amino |
| acid anhydride, | primary amino, secondary amino, hydroxyl |
| acid halide. | |
| isothiocyanate | amino groups |
| vinylsulfone | amino groups |
| dichlorotriazine | amino groups |
| haloacetamide, | thiol, imidazole, hydroxyl, amines, |
| maleimide | thiophosphate |
| carbodiimide | carboxylic acids |
| hydrazine, hydrazide | carbonyl including aldehyde and ketone |
| phosphoramidite | hydroxyl groups |

By the term “activated ester” or “active ester” is meant an ester derivative of the carboxylic acid which is designed to be a better leaving group, and hence permit more facile reaction with nucleophile, such as amines. Examples of suitable active esters are: N-hydroxysuccinimide (NHS), pentafluorophenol, pentafluorothiophenol, para-nitrophenol and hydroxybenzotriazole. Preferred active esters are N-hydroxysuccinimide or pentafluorophenol esters.

Examples of functional groups present in BTM such as proteins, peptides, nucleic acids carbohydrates and the like, include: hydroxy, amino, sulfydryl, carbonyl (including aldehyde and ketone) and thiophosphate. Suitable Q^{a} groups may be selected from: carboxyl; activated esters; isothiocyanate; maleimide; haloacetamide; hydrazide; vinylsulfone, dichlorotriazine and phosphoramidite. Preferably, Q^{a} is: an activated ester of a carboxylic acid, an isothiocyanate, a maleimide or a haloacetamide.

When the complementary group is an amine or hydroxyl, Q^{a} is preferably an activated ester, with preferred such esters as described above. A preferred such substituent on the Bzp^{M} is the activated ester of a 5-carboxypentyl group. When the complementary group is a thiol, Q^{a} is preferably a maleimide or iodoacetamide group.

General methods for conjugation of dyes to biological molecules are described by Licha et al [Topics Curr. Chem., 222, 1-29 (2002); Adv. Drug Deliv. Rev., 57, 1087-1108 (2005)]. Peptide, protein and oligonucleotide substrates for use in the invention may be labelled at a terminal position, or alternatively at one or more internal positions. For reviews and examples of protein labelling using fluorescent dye labelling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

Preferably, the method of preparation of the imaging agent comprises either:
(i) reaction of an amine functional group of a BTM with a compound of formula J¹-(L)ₙ-[Bzp^{M}]; or
(ii) reaction of a carboxylic acid or activated ester functional group of a BTM with a compound of formula J²-(L)ₙ-[Bzp^{M}];
(iii) reaction of a thiol group of a BTM with a compound of formula

J³-(L)ₙ-[Bzp^{M}];
wherein BTM, M^{IG}, L, n and Bzp^{M} are as defined above, and
J¹ is a carboxylic acid, activated ester, isothiocyanate or thiocyanate group;
J² is an amine group;
J³ is a maleimide group.

J² is preferably a primary or secondary amine group, most preferably a primary amine group. In step (iii), the thiol group of the BTM is preferably from a cysteine residue.

In steps (i) to (iii), the BTM may optionally have other functional groups which could potentially react with the Bzp^{M} derivative, protected with suitable protecting groups so that chemical reaction occurs selectively at the desired site only. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). Suitable thiol protecting groups are Trt (Trityl), Acm (acetamidomethyl), t-Bu (tert-butyl), tert-Butylthio, methoxybenzyl, methylbenzyl or Npys (3-nitro-2-pyridine sulfenyl). The use of further protecting groups are described in ‘Protective Groups in Organic Synthesis’, Theodora W. Greene and Peter G. M. Wuts, (John Wiley & Sons, 1991). Preferred amine protecting groups are Boc and Fmoc, most preferably Boc. Preferred amine protecting groups are Trt and Acm.

Benzopyrylium dyes (Bzp^{M}) functionalised suitable for conjugation to BTM are commercially available from Dyomics (Dyomics GmbH, Winzerlaer Str. 2A, D-07745 Jena, Germany; www.dyomics.com), where the reactive functional group (Q^{a}) is NHS ester, maleimide, amino or carboxylic acid. Precursors suitable for the synthesis of benzopyrylium dyes can also be prepared as described in U.S. Pat. No. 5,405,976. Methods of conjugating optical reporter dyes, to amino acids and peptides are described by Licha (vide sepia), as well as Flanagan et al [Bioconj. Chem., 8, 751-756 (1997)]; Lin et al, [ibid, 13, 605-610 (2002)] and Zaheer [Mol. Imaging, 1(4), 354-364 (2002)]. Methods of conjugating the linker group (L) to the BTM employ analogous chemistry to that of the dyes alone (see above), and are known in the art.

In a second aspect, the present invention provides a kit for the preparation of the pharmaceutical composition of the first aspect, wherein said kit comprises the conjugate of Formula I in sterile, solid form such that, upon reconstitution with a sterile supply of the biocompatible carrier, dissolution occurs to give the desired pharmaceutical composition. The “conjugate” and “biocompatible carrier”, together with preferred embodiments thereof are as described in the first aspect.

For the kit, the conjugate, plus other optional excipients as described above, may be provided as a lyophilised powder in a suitable vial or container. The powder is then designed to be reconstituted with the desired biocompatible carrier to give the pharmaceutical composition in a sterile, apyrogenic form which is ready for mammalian administration.

A preferred sterile, solid form of the conjugate is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

In a third aspect, the present invention provides a conjugate of Formula

[BTM′]-(L)ₙ-Bzp^{M} (I)
where: L and n are as defined for the first aspect, and Bzp^{M} is of Formula II as defined above; BTM′ is a BTM as defined in the first aspect, which is synthetic and is chosen from:
   (i) a 3-100 mer peptide;
   (ii) an enzyme substrate, enzyme antagonist or enzyme inhibitor;
   (iii) a receptor-binding compound;
   (iv) an oligonucleotide;
   (v) an oligo-DNA or oligo-RNA fragment.

The term ‘synthetic’ has the definition given above. Preferred embodiments of the Bzp^{M} of Formula II in the conjugate are as described in the first aspect above. Preferred aspects of BTM′ of (i)-(v) are as described in the first aspect for those types of BTM. BTM′ is preferably a 3-100 mer peptide.

The conjugates of the third aspect are useful in the preparation of the imaging agent pharmaceutical compositions of the invention. The conjugates can be prepared as described in the first aspect.

In a fourth aspect, the present invention provides a method of in vivo optical imaging of the mammalian body which comprises use of the pharmaceutical composition of the first aspect to obtain images of sites of BTM localisation in vivo.

The term “optical imaging” is as defined in the first aspect (above).

In the method of the fourth aspect, the imaging agent pharmaceutical composition has preferably been previously administered to said mammalian body. By “previously administered” is meant that the step involving the clinician, wherein the imaging agent is given to the patient eg. as an intravenous injection, has already been carried out prior to imaging. This embodiment includes the use of the conjugate as defined in the first aspect in the manufacture of a diagnostic agent for optical imaging in vivo of disease states of the mammalian body where the BTM is implicated.

A preferred optical imaging method of the fourth aspect is Fluorescence Reflectance Imaging (FRI). In FRI, the imaging agent of the present invention is administered to a subject to be diagnosed, and subsequently a tissue surface of the subject is illuminated with an excitation light—usually continuous wave (CW) excitation. The light excites the Bzp^{M} dye of the imaging agent. Fluorescence from the imaging agent, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The imaging agent is designed to concentrate in the disease area, producing higher fluorescence intensity. Thus the disease area produces positive contrast in a fluorescence intensity image. The image is preferably obtained using a CCD camera or chip, such that real-time imaging is possible.

The wavelength for excitation varies depending on the particular Bzp^{M} dye used, but is typically in the range 500-1200 nm for dyes of the present invention. The apparatus for generating the excitation light may be a conventional excitation light source such as: a laser (e.g., ion laser, dye laser or semiconductor laser); halogen light source or xenon light source. Various optical filters may optionally be used to obtain the optimal excitation wavelength. A preferred FRI method comprises the steps as follows:
(i) a tissue surface of interest within the mammalian body is illuminated with an excitation light;
(ii) fluorescence from the imaging agent, which is generated by excitation of the Bzp^{M}, is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

In step (i), the excitation light is preferably continuous wave (CW) in nature. In step (iii), the light detected is preferably filtered. An especially preferred FRI method is fluorescence endoscopy.

An alternative imaging method of the sixth aspect uses FDPM (frequency-domain photon migration). This has advantages over continuous-wave (CW) methods where greater depth of detection of the dye within tissue is important [Sevick-Muraca et al, Curr. Opin. Chem. Biol., 6, 642-650 (2002)]. For such frequency/time domain imaging, it is advantageous if the Bzp^{M} has fluorescent properties which can be modulated depending on the tissue depth of the lesion to be imaged, and the type of instrumentation employed.

The FDPM method is as follows:
(a) exposing light-scattering biological tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to uptake of the imaging agent and preferably further comprises mapping a number of quantities corresponding to adsorption and scattering coefficients of the tissue before administration of the imaging agent. The fluorescence characteristic of step (c) preferably corresponds to at least one of fluorescence lifetime, fluorescence quantum efficiency, fluorescence yield and imaging agent uptake. The fluorescence characteristic is preferably independent of the intensity of the emission and independent of imaging agent concentration.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The optical imaging of the fourth aspect is preferably used to help facilitate the management of a disease state of the mammalian body. By the term “management” is meant use in the: detection, staging, diagnosis, monitoring of disease progression or the monitoring of treatment. The disease state is suitably one in which the BTM of the imaging agent is implicated. Imaging applications preferably include camera-based surface imaging, endoscopy and surgical guidance. Further details of suitable optical imaging methods have been reviewed by Sevick-Muraca et al [Curr. Opin. Chem. Biol., 6, 642-650 (2002)].

In a fifth aspect, the present invention provides a method of detection, staging, diagnosis, monitoring of disease progression or monitoring of treatment of a disease state of the mammalian body which comprises the in vivo optical imaging method of the fourth aspect.

The invention is illustrated by the non-limiting Examples detailed below. Example 1 provides the synthesis of a biological targeting peptide (Peptide 1), which binds to cMet. Example 2 provides methods of conjugating Bzp^{M} dyes of the invention to peptides, in particular Peptide 1. Example 3 provides data demonstrating that the peptide conjugates of Peptide 1 of the invention retain affinity for cMet, i.e. that the conjugated dye does not interfere with the biological binding and selectivity. Appropriate low binding to human serum albumin and high stability in plasma were demonstrated. Example 4 shows that the peptide conjugates of the invention exhibit useful tumour:background ratios in an animal model of colorectal cancer. Example 5 describes the use of predictive software for the dyes of the invention, and demonstrates that the dyes of the invention lack potentially dangerous metabolites in vivo. Example 6 describes the toxicity testing of Compound 6, showing that the anticipated clinical dose was well tolerated and without any drug substance related adverse effects.

TABLE 2
Structures of Benzopyrylium dyes of the Examples.

| | DY-630 | DY-631 | DY-633 | DY-650 | DY-651 | DY-652 |
|---|---|---|---|---|---|---|
| Formula | IIId | IIId | IIId | IIIe | IIIe | IIIe |
| R¹⁷ | Et | Et | Et | — | — | — |
| R¹⁸ | Et | Et | R^{d} | — | — | — |
| R¹⁹ | Bu^{t} | Bu^{t} | Bu^{t} | Bu^{t} | Bu^{t} | Bu^{t} |
| R²⁰ | CH₃ | R^{e} | CH₃ | CH₃ | R^{e} | R^{e} |
| R²¹ | R^{f} | R^{d} | R^{f} | R^{f} | R^{d} | R^{d} |
| R²² | — | — | — | Et | Et | R^{d} |
| X² | — | — | — | CH₃ | CH₃ | CH₃ |
| X³ | — | — | — | CH₃ | CH₃ | CH₃ |
| X⁴ | — | — | — | CH₃ | CH₃ | CH₃ |

where: R^{d} is —(CH₂)₃SO₃H, R^{e} is —(CH₂)₃CO₂H and R^{f} is —(CH₂)₅CO₂H.

DY-752 has the same rings and substituent pattern as DY-652, but has a pentamethine linkage (i.e. w=2 and R⁵═H) in place of the trimethine linkage of DY-652.

### Abbreviations.

Conventional 3-letter and single letter amino acid abbreviations are used.

### Acm: Acetamidomethyl

### ACN: Acetonitrile

Boc: tert-Butyloxycarbonyl

### DMF: N,N′-Dimethylformamide

### DMSO: Dimethylsulfoxide

### Fmoc: 9-Fluorenylmethoxycarbonyl

HCl: Hydrochloric acid
HPLC: High performance liquid chromatography
HSPyU O—(N-succinimidyl)-N,N,N′,N′-tetramethyleneuronium hexafluorophosphate

### Ile: Isoleucine

LC-MS: Liquid chromatography mass spectroscopy

### NHS: N-hydroxy-succinimide

### NMM: N-Methylmorpholine

NMP: 1-Methyl-2-pyrrolidinone
Pbf: 2,2,4,6,7-Pentamethyldihydrobenzofuran-5-sulfonyl
PBS: Phosphate-buffered saline.
TFA: Trifluoroacetic acid

### Trt: Trityl

TSTU: O—(N-Succinimidyl)-N,N,N′,N′-tetramethyluronium tetrafluoroborate

### Example 1

### Synthesis of Peptide 1

A 26-mer bicyclic peptide having 2 Cys-Cys bonds (Cys4-16 and 6-14) having the following sequence was used:

Ac-Ala-Gly-Ser-Cys-Tyr-Cys-Ser-Gly-Pro-Pro-Arg-
Phe-Glu-Cys-Trp-Cys-Tyr-Glu-Thr-GlU-Gly-Thr-Gly-
Gly-Gly-Lys-NH₂ (“Peptide I”).

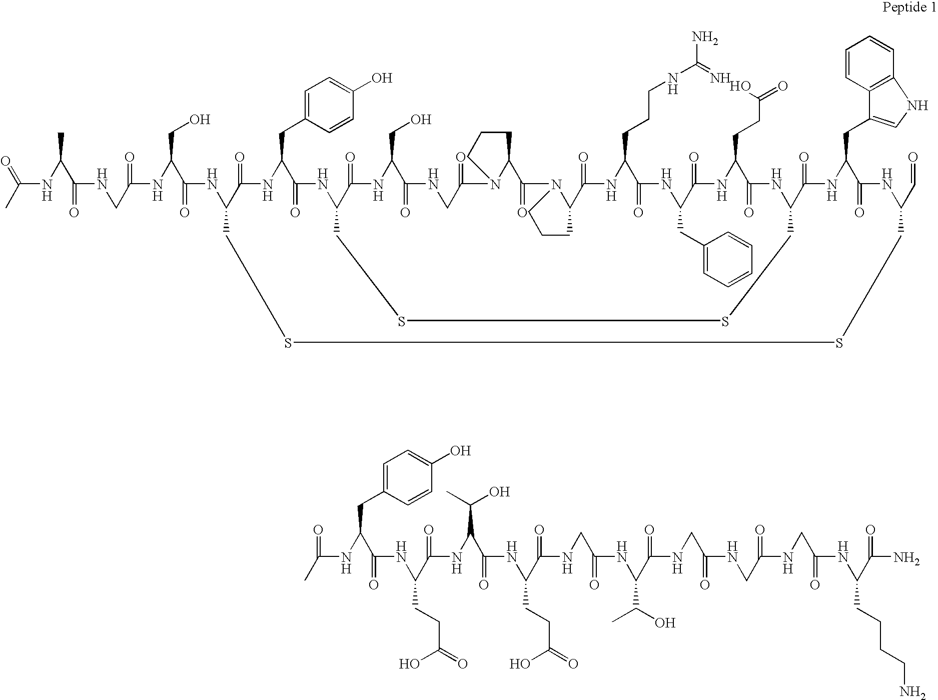

### Step (a): Synthesis of Protected Linear Precursor of Peptide 1

The precursor linear peptide has the sequence:

Ac-Ala-Gly-Ser-Cys-Tyr-Cys(Acm)-Ser-Gly-Pro-Pro-
Arg-Phe-Glu-Cys(Acm)-Trp-Cys-Tyr-G1u-Thr-Glu-Gly-
Thr-Gly-Gly-Gly-LYS-NH₂.

The peptidyl resin H-Ala-Gly-Ser(tBu)-Cys(Trt)-Tyr(tBu)-Cys(Acm)-Ser(tBu)-Gly-Pro-Pro-Arg(Pbf)-Phe-Glu(OtBu)-Cys(Acm)-Trp(Boc)-Cys(Trt)-Tyr(tBu)-Glu(OtBu)-Thr(ψ^{Me.Me}pro)-Glu(OtBu)-Gly-Thr(tBu)-Gly-Gly-Gly-Lys(Boc)-Polymer was assembled on an Applied Biosystems 433A peptide synthesizer using Fmoc chemistry starting with 0.1 mmol Rink Amide Novagel resin. An excess of 1 mmol pre-activated amino acids (using HBTU) was applied in the coupling steps. Glu-Thr pseudoproline (Novabiochem 05-20-1122) was incorporated in the sequence. The resin was transferred to a nitrogen bubbler apparatus and treated with a solution of acetic anhydride (1 mmol) and NMM (1 mmol) dissolved in DCM (5 mL) for 60 min. The anhydride solution was removed by filtration and the resin washed with DCM and dried under a stream of nitrogen.

The simultaneous removal of the side-chain protecting groups and cleavage of the peptide from the resin was carried out in TFA (10 mL) containing 2.5% TIS, 2.5% 4-thiocresol and 2.5% water for 2 hours and 30 min. The resin was removed by filtration, TFA removed in vacuo and diethyl ether added to the residue. The formed precipitate was washed with diethyl ether and air-dried affording 264 mg of crude peptide.

Purification by preparative HPLC (gradient: 20-30% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 30 min) of the crude peptide afforded 100 mg of pure Peptide 1 linear precursor. The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 6.54 min). Further product characterisation was carried out using electrospray mass spectrometry (MH₂²⁺ calculated: 1464.6, MH₂²⁺ found: 1465.1).

### Step (b): Formation of Cys4-16 Disulfide Bridge

Cys4-16; Ac-Ala-Gly-Ser-Cys-Tyr-Cys(Acm)-Ser-Gly-
Pro-Pro-Arg-Phe-Glu-Cys(Acm)-Trp-Cys-Tyr-Glu-Thr-
Glu-Gly-Thr-Gly-Gly-Gly-Lys-NH₂.

The linear precursor from step (a) (100 mg) was dissolved in 5% DMSO/water (200 mL) and the solution adjusted to pH 6 using ammonia. The reaction mixture was stirred for 5 days. The solution was then adjusted to pH 2 using TFA and most of the solvent removed by evaporation in vacuo. The residue (40 mL) was injected in portions onto a preparative HPLC column for product purification.

Purification by preparative HPLC (gradient: 0% B for 10 min, then 0-40% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 44 min) of the residue afforded 72 mg of pure Peptide 1 monocyclic precursor.

The pure product (as a mixture of isomers P1 to P3) was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 5.37 min (P1); 5.61 min (P2); 6.05 min (P3)). Further product characterisation was carried out using electrospray mass spectrometry (MH₂²⁺ calculated: 1463.6, MH₂²⁺ found: 1464.1 (P1); 1464.4 (P2); 1464.3 (P3)).

### Step (c): Formation of Cys6-14 Disulfide Bridge (Peptide 1)

The monocyclic precursor from step (b) (72 mg) was dissolved in 75% AcOH/water (72 mL) under a blanket of nitrogen. 1 M HCl (7.2 mL) and 0.05 M I₂ in AcOH (4.8 mL) were added in that order and the mixture stirred for 45 min. 1 M ascorbic acid (1 mL) was added giving a colourless mixture. Most of the solvents were evaporated in vacuo and the residue (18 mL) diluted with water/0.1% TFA (4 mL) and the product purified using preparative HPLC. Purification by preparative HPLC (gradient: 0% B for 10 min, then 20-30% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 43-53 min) of the residue afforded 52 mg of pure Peptide 1. The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 6.54 min). Further product characterisation was carried out using electrospray mass spectrometry (MH₂²⁺ calculated: 1391.5, MH₂²⁺ found: 1392.5).

### Example 2

### Synthesis of Peptide Conjugates of Benzopyrylium Dyes

### General Conjugation Method.

To a solution of Peptide 1 (from Example 1; 4 mg, 1.4 μmol) in DMF (0.5 mL) was added a solution of Bzp^{M} NHS ester (1 mg, 1 μmol) and sym.-collidine (8 μL, 60 μmol) in DMF (0.5 mL). The reaction mixture was heated (microwave assisted) at 60° C. for 1 hr, then at RT overnight. The reaction mixture was then diluted with 20% ACN/water/0.1 TFA (7 mL) and the product purified using preparative HPLC.

### Purification and Characterisation.

Purification by preparative HPLC (gradient: 20-40% B over 40 min where A=H₂O/0.1% TFA and B=ACN/0.1% TFA, flow rate: 10 mL/min, column: Phenomenex Luna 5μ C18 (2) 250×21.2 mm, detection: UV 214 nm) of the crude peptide afforded pure [Peptide 1]-Bzp^{M} conjugate. The pure product was analysed by analytical HPLC (gradient: 10-40% B over 5 min where A=H₂O/0.1% TFA and B=ACN/0.1% TFA, flow rate: 0.6 mL/min, column: Phenomenex Luna 3μ C18 (2) 20×2 mm, detection: UV 214 nm). Further product characterisation was carried out using electrospray mass spectrometry.

The compounds prepared are given in Table 3:

TABLE 3
Peptide-dye conjugates of Peptide 1.

| | | Synthesis | MS found (MS |
|---|---|---|---|
| Compound | Bzp^{M} | yield | theoretical) |
| 1 | DY-630 | 2.1 mg | 1700.7 (MH²⁺ 1699.7) |
| | | (44%) | |
| 2 | DY-631 | 2.5 mg | 1740.2 (MH²⁺ 1739.7) |
| | | (60%) | |
| 3 | DY-633 | 2.5 mg | 1747.4 (MH²⁺ 1746.7) |
| | | (60%) | |
| 4 | DY-650 | 3.1 mg | 1726.1 (MH²⁺ 1725.7) |
| | | (69%) | |
| 5 | DY-651 | 3.0 mg | 1766.4 (MH²⁺ 1765.7) |
| | | (77%) | |
| 6 | DY-652 | 3.3 mg | 1813.5 (MH²⁺ 1812.7) |
| | | (91%) | |
| 7 | DY-752 | 1.8 mg | 1825.9 (MH²⁺ 1825.7) |
| | | (45%) | |

### Example 3

### In Vitro Fluorescence Polarisation Assay

Fluorescence polarisation assay was used to examine the affinity binding of the imaging agent towards the cMet target as well as the binding properties related to plasma proteins. The principle of the fluorescence polarisation method can briefly be described as follows:

Monochromatic light passes through a horizontal polarizing filter and excites fluorescent molecules in the sample. Only those molecules that oriented properly in the vertically polarized plane adsorb light, become excited, and subsequently emit light. The emitted light is measured in both horizontal and vertical planes. The anisotropy value (A), is the ratio between the light intensities following the equation

$$A = \frac{\begin{matrix}Intensity with horizontal polarizer - \\ Intensity with vertical polarizer\end{matrix}}{\begin{matrix}Intensity with horizontal polarizer + \\ {2}^{*} Intensity with vertical polarizer\end{matrix}}$$

The fluorescence anisotropy measurements were performed in 384-well microplates in a volume of 10 μL in binding buffer (PBS, 0.01% Tween-20, pH 7.5) using a Tecan Safire fluorescence polarisation plate reader (Tecan, US) at Ex 635/Em 678 nm. The concentration of dye-labelled peptide was held constant (5 nM) and the concentration of the human c-Met/Fc chimera (R&D Systems) was varied from 0-250 nM. Binding mixtures were equilibrated in the microplate for 10 min at 30° C. The observed change in anisotropy was fitted to the equation:

$${r}_{obs} = {r}_{free} + ( {r}_{bound} - {r}_{free} ) \frac{\begin{matrix}( {K}_{D} + cMet + P ) - \\ \sqrt{( {K}_{D} + cMet + P ) 2 - 4 \cdot cMet \cdot P}\end{matrix}}{2 \cdot P}$$

where robs is the observed anisotropy, rfree is the anisotropy of the free peptide, rbound is the anisotropy of the bound peptide, K_{d} is the dissociation constant, cMet is the total c-Met concentration, and P is the total dye-labelled peptide concentration. The equation assumes that the synthetic peptide and the receptor form a reversible complex in solution with 1:1 stoichiometry. Data fitting was done via nonlinear regression using SigmaPlot software to obtain the K_{d} value (one-site binding).

Compounds 1 to 6 were tested for binding towards human c-Met (Fc chimera). The to results (see Table 4) showed a K_{d} of nM for the binding of all compounds tested to human c-Met.

The change of the polarization value was used to assess the binding of the Compound to human serum albumin as a low change of polarisation value is associated to low binding being appropriate for in-vivo use. The plasma protein binding (PPB) was confirmed with Biacore measurements. The stability of the imaging agent in plasma was confirmed by measuring the amount of the Compound left after incubation in mouse plasma for 2 hours at 37° C.

TABLE 4
in vitro properties of Compounds 1-6.

| | | PPB (% change | Binding human | Mouse plasma |
|---|---|---|---|---|
| Com- | Affinity | in polarisation | serum albumin | stability |
| pound | (Kd, nM) | value) | (Biacore) | (2 h, 37° C.) |
| 1 | 2.2 | 36 | Very high | >95% |
| 2 | 0.5 | 33 | Very low | >95% |
| 3 | 0.5 | 27 | Low | >95% |
| 4 | 3.2 | 55 | Very high | >95% |
| 5 | 2.2 | 49 | Medium | >95% |
| 6 | 0.9 | 46 | Very low | >95% |

### Example 4

### In Vivo Testing of Compounds 2 to 6

(a) Animal Model.

Female BALB c/A nude (Born) mice were used in the study. The use of the animals was approved by the local ethics committee. BALB c/A nude is an inbred immunocompromised mouse strain with a high take rate for human tumours as compared to other nude mice strains. The mice were 8 weeks old upon arrival and with a body weight of approx. 20 grams at the start of the study. The animals were housed in individually ventilated cages (IVC, Scanbur BK) with HEPA filtered air. The animals had ad libitum access to “Rat and Mouse nr. 3 Breeding” diet (Scanbur BK) and tap water acidified by addition of HCl to a molar concentration of 1 mM (pH 3.0).

The colon cancer cell HT-29 is derived from human colon carcinomas and is reported to express c-Met according to Zeng et al [Clin. Exp. Metastasis, 21, 409-417. (2004)]. The cell line was proven to be tumorigenic when inoculated subcutaneously into nude mice [Flatmark et al, Eur. J. Cancer 40, 1593-1598 (2004)].

HT-29 cells were grown in McCoy's 5a medium (Sigma #M8403) supplemented with 10% fetal bovine serum and penicillin/streptomycin. Stocks were made at passage number four (P4) and frozen down for storage in liquid nitrogen at 10⁷ cells/vial in the respective culture media containing 5% DMSO. On the day of the transplantation, the cells were thawed quickly in 37° C. water bath (approx. 2 min), washed and resuspended in PBS/2% serum (centrifugation at 1200 rpm for 10 min). Thorough mixing of cells in the vials was ensured every time the cells were aspirated into the dosing syringe. A volume of 0.1 ml of cell suspension was injected s.c. at the shoulder and at the back using a fine bore needle (25 G). The animals were then returned to their cages and the tumours were allowed to grow for 13-17 days. The animals were allowed an acclimatisation period of at least 5 days before the inoculation procedure.

(b) Procedure.

All test substances were reconstituted with PBS from freeze-dried powder. A small stack of white printer paper was imaged to obtain a flat field image which was used to correct for illumination inhomogeneities.

For immobilisation during the optical imaging procedure, the animals were anaesthetized in a coaxial open mask to light surgical level anaesthesia with Isoflurane (typically 1.3-2%), using oxygen as the carrier gas. A small piece of skin (3-5 mm) was removed over parts of the tumour and adjacent muscle using a surgical forceps and fine scissors while the animal was anaesthetized. This was done to measure the signal from tumour and muscle without interference from the overlying skin tissue. The wound was covered by applying a liquid, non-fluorescent bandage spray (3M, MN, USA).

The respiration and body temperature of the animal was monitored with a BioVet system (m2m Imaging Corp, NJ, USA) using a pneumatic sensor underneath the animal and a rectal temperature probe. The BioVet system also supplied external heating using a heating mat set to 40° C. to sustain normal body temperature for the duration of the imaging procedure (2 hours). A Venflon catheter was placed in the tail vein for contrast agent administration. Each animal was given one contrast agent injection. The injected volume was 0.1 ml of test compound followed immediately by a 0.2 ml saline flush. Fluorescence images were acquired just prior to injection and then every 30 seconds for 2 hours.

(c) Imaging.

Imaging was performed through a clinical laparoscope adapted to use a light source to excite the reporter and a filtering system to extract the fluorescence component. A 635 nm laser was used for excitation of the reporter molecule. A Hamamatsu ORCA ERG CCD camera was used as the detector. The camera was operated in 2×2 binning mode with 0 gain. Standard exposure time for colon imaging was 4 s. The intensity distribution in the image was corrected for illumination inhomogeneities through system calibration data. A target to background ratio was computed from regions of interest placed over the exposed tumour and normal muscle background.

(d) Results.

The test Compounds had the following average tumour:muscle ratios (Table 5):

TABLE 5
tumour:muscle ratios of Compounds 2 to 6.

| | Average tumour:muscle |
|---|---|
| Compound | ratio (2 hours p.i.) |
| 2 | 2.40 |
| 3 | 1.67 |
| 4 | 1.52 |
| 5 | 1.22 |
| 6 | 1.57 |

### Example 5

### Metabolism and Toxicity Prediction

The software tools Derek and Meteor were obtained from Lhasa Ltd (22-23 Blenheim Terrace, Leeds LS2 9HD, UK). Derek is used for predicting toxicity of new chemical entities based on known structure-dependent toxicity. Similarly, Meteor predicts likely metabolites of novel chemicals. Both tools are based on published and unpublished (but verified) data for chemical compounds. The chemical structure of dye DY-652 was input. No potentially dangerous metabolites in vivo were predicted.

### Example 6

### Toxicity Testing of Compound 6

A limited acute dose toxicity study was conducted to investigate the tolerance of Compound 6 at 100 times the preclinical imaging dose (50 nmol/kg body weight).

The compound was injected intravenously in male rats, and the animals were sacrificed at 1, 14, 21 and 28 days post injection (p.i.). At necropsy, the major organs were inspected for gross pathology, and the kidneys were taken into neutral buffered formalin for subsequent histomorphological evaluation. A weak blue colouration of the skin and a moderate blue colouration of the urine were observed immediately after injection, which disappeared within 1 day p.i. At necropsy, the kidneys were diffusely green on day 1 p.i. Light microscopy showed no Compound 6-related findings in the kidneys. The other minor changes seen were incidental and common in young adult laboratory rats. Strong fluorescence staining of blood vessels in the kidney was observed on day 1 p.i. The staining was reduced by day 14 p.i. and was not discernible from control on day 21 p.i.

No evidence of degeneration, necrosis or inflammation was noted in any of the treated animals, suggesting that the nephrotoxicity of the compound is low. It was concluded that a single intravenous administration of Compound 6 to male rats at 100 times the anticipated clinical dose was well tolerated and without any drug substance related adverse effects.

What is claimed is:

1. A pharmaceutical composition which comprises an imaging agent suitable for in vivo optical imaging of the mammalian body, together with a biocompatible carrier, said composition being in a form suitable for mammalian administration, wherein said imaging agent comprises a conjugate of Formula I:
[BTM]-(L)ₙ-Bzp^{M} (I)
where:
BTM is a biological targeting moiety;
n is an integer of value 0 or 1;
L is a synthetic linker group of formula -(A)ₘ- wherein m is an integer of value 1 to 20, and each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block; wherein each R is independently chosen from H, C₁₋₄ alkyl, C₂₋₄ alkenyl, C₂₋₄ alkynyl, C₁₋₄ alkoxyalkyl or C₁₋₄ hydroxyalkyl;
Bzp^{M} is a benzopyrylium dye of Formula II: 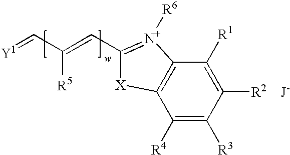
where:
Y¹ is a group of Formula Y^{a} or Y^{b} 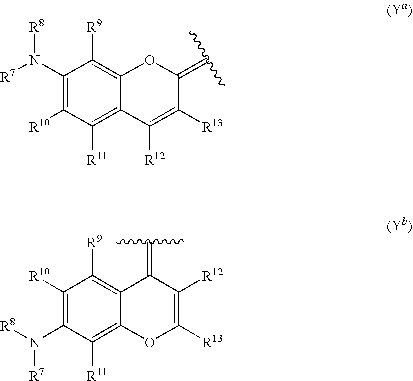
R¹-R⁴ and R⁹-R¹³ are independently selected from H, —SO₃M¹, Hal, R^{a} or C₃₋₁₂ aryl, where each M¹ is independently H or B^{c}, and B^{c} is a biocompatible cation;
R⁵ is H, C₁₋₄ alkyl, C₁₋₆ carboxyalkyl, C₃₋₁₂ arylsulfonyl, Cl, or R⁵ together with one of R⁶, R¹⁴, R¹⁵ or R¹⁶ may optionally form a 5- or 6-membered unsaturated aliphatic, unsaturated heteroaliphatic or aromatic ring;
R⁶ and R¹⁶ are independently R^{a} groups;
R⁷ and R⁸ are independently C₁₋₄ alkyl, C₁₋₄ sulfoalkyl or C₁₋₆ hydroxyalkyl or optionally together with one or both of R⁹ and/or R¹⁰ may form a 5- or 6-membered N-containing heterocyclic or heteroaryl ring;
X is —CR¹⁴R¹⁵—, —O—, —S—, —Se—, —NR¹⁶— or —CH═CH—, where R¹⁴ to R¹⁶ are independently R^{a} groups;
R^{a} is C₁₋₄ alkyl, C₁₋₄ sulfoalkyl, C₁₋₆ carboxyalkyl or C₁₋₆ hydroxyalkyl;
w is 1 or 2;
J is a biocompatible anion;
with the proviso that Bzp^{M} comprises at least one sulfonic acid substituent chosen from the R¹ to R¹⁶ groups.

2. The composition of claim 1, where Bzp^{M} is of Formula IIa:

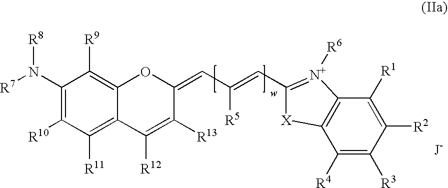

3. The composition of claim 1, where Bzp^{M} is of Formula IIb:

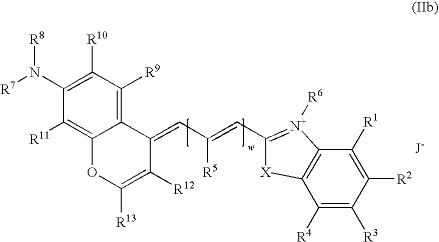

4. The composition of claim 1, where the Bzp^{M} comprises 2 to 4 sulfonic acid substituents.

5. The composition of claim 1, where the Bzp^{M} comprises at least one C₁₋₄ sulfoalkyl substituent.

6. The composition of claim 5, where the sulfoalkyl substituent is of formula
—(CH₂)ₖSO₃M¹, where M¹ is H or B^{c}, and k is an integer of value 1 to 4.

7. The composition of claim 1, where w is 1.

8. The composition of claim 1, where R⁵ is H.

9. The composition of claim 1, where X is —CR¹⁴R¹⁵—.

10. The composition of claim 1, where Bzp^{M} is of Formula III: 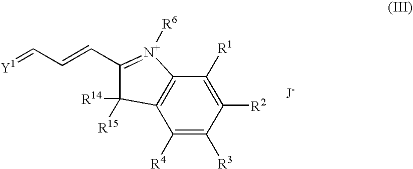
where Y¹, R¹-R⁴, R⁶, R¹⁴, R¹⁵ and J are as defined in claim 1.

11. The composition of claim 10, where Bzp^{M} is of Formula IIIc, IIId or IIIe: 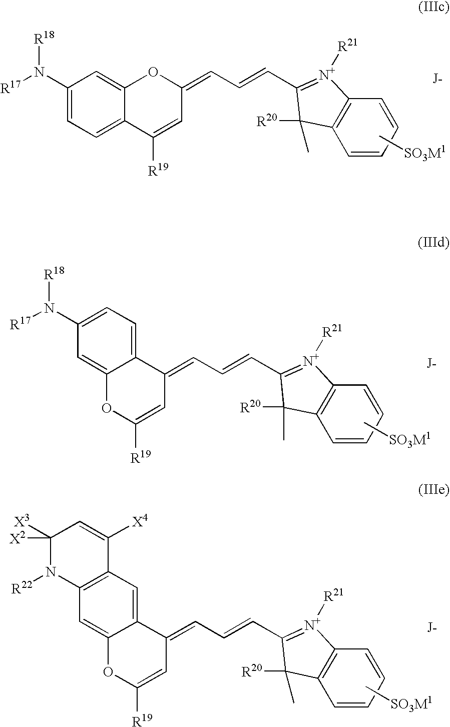
where:
M¹ is independently H or B^{c}, and B^{c} is a biocompatible cation;
R¹⁷ and R¹⁸ are independently chosen from C₁₋₄ alkyl or C₁₋₄ sulfoalkyl;
R¹⁹ is H or C₁₋₄ alkyl;
R²⁰ is C₁₋₄ alkyl, C₁₋₄ sulfoalkyl or C₁₋₆ carboxyalkyl;
R²¹ is C₁₋₄ sulfoalkyl or C₁₋₆ carboxyalkyl;
R²² is C₁₋₄ alkyl, C₁₋₄ sulfoalkyl or C₁₋₆ carboxyalkyl;
X², X³ and X⁴ are independently H or C₁₋₄ alkyl.

12. The composition of claim 1, where BTM is chosen from:
(i) a 3-100 mer peptide;
(ii) an enzyme substrate, enzyme antagonist or enzyme inhibitor;
(iii) a receptor-binding compound;
(iv) an oligonucleotide; and
(v) an oligo-DNA or oligo-RNA fragment.

13. The composition of claim 12, where BTM is a 3-100 mer peptide.

14. The composition of claim 13, where said conjugate of Formula I is of Formulae IVa or IVb:
[Bzp^{M}]-(L)ₙ-[BTM]-Z² (IVa);
Z¹-[BTM]-(L)ₙ-[Bzp^{M}] (IVb);
where:
Z¹ is attached to the N-terminus of the BTM peptide, and is H or M^{IG};
Z² is attached to the C-terminus of the BTM peptide and is OH, OB^{c}, or M^{IG},
where B^{c} is a biocompatible cation, and
M^{IG} is a metabolism inhibiting group which is a biocompatible group which inhibits or suppresses enzyme metabolism of the BTM peptide.

15. The composition of claim 14, where each of Z¹ and Z² is independently M^{IG}.

16. The composition of claim 1, which has a dosage suitable for a single patient and is provided in a suitable syringe or container.

17. A kit for the preparation of the pharmaceutical composition of claim 1, which comprises the conjugate of Formula I as defined in claim 1 in sterile, solid form such that upon reconstitution with a sterile supply of the biocompatible carrier, dissolution occurs to give the desired pharmaceutical composition.

18. The kit of claim 17, where the sterile, solid form is a lyophilised solid.

19. A conjugate of Formula I:
[BTM′]-(L)ₙ-Bzp^{M} (I)
where: L and n are as defined in claim 1, Bzp^{M} is as defined in claim 1, and BTM′ is a biological targeting moiety which is synthetic and chosen from:
(i) a 3-100 mer peptide;
(ii) an enzyme substrate, enzyme antagonist or enzyme inhibitor;
(iii) a receptor-binding compound;
(iv) an oligonucleotide; and
(v) an oligo-DNA or oligo-RNA fragment.

20. A method of in vivo optical imaging of the mammalian body which comprises use of the pharmaceutical composition of claim 1 to obtain images of sites of localisation of the BTM in vivo.

21. The method of claim 20, where the pharmaceutical composition has been previously administered to said mammalian body.

22. The method of claim 21, which comprises the steps of:
(i) a tissue surface of interest within the mammalian body is illuminated with an excitation light;
(ii) fluorescence from the imaging agent, which is generated by excitation of the Bzp^{M} is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

23. The method of claim 22 where the excitation light of step (i) is continuous wave (CW) in nature.

24. The method of claim 21 which comprises:
(a) exposing light-scattering biologic tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

25. The method of claim 20, where the optical imaging method comprises fluorescence endoscopy.

26. The method of claim 20, where the in vivo optical imaging is used to assist in the detection, staging, diagnosis, monitoring of disease progression or monitoring of treatment of a disease state of the mammalian body.

27. A method of detection, staging, diagnosis, monitoring of disease progression or monitoring of treatment of a disease state of the mammalian body which comprises the in vivo optical imaging method of claim 20.
